(19) United States
(12) Patent Application Publication
(10) Pub. No.: US 2026/0014155 A1
(43) Pub. Date: Jan. 15, 2026

(54) TICAGRELOR SOLID DISPERSION, METHOD FOR PREPARING SAME, AND USE THEREOF

(71) Applicants: WISDOM PHARMACEUTICAL CO., LTD., Nantong, Jiangsu (CN); WISDOM PHARMACEUTICAL CO., LTD. GUANGZHOU BRANCH, Guangzhou, Guangdong (CN)

(72) Inventors: Ping ZOU, Nantong, Jiangsu (CN); Yiguang QI, Guangzhou, Guangdong (CN); Ni WANG, Guangzhou, Guangdong (CN); Diling LIU, Guangzhou, Guangdong (CN); Wenjie YANG, Guangzhou, Guangdong (CN)

(21) Appl. No.: 18/994,956

(22) PCT Filed: May 22, 2023
(86) PCT No.: PCT/CN2023/095585

§ 371 Date: Jan. 15, 2025

(30) Foreign Application Priority Data
Jul. 27, 2022 (CN) 202210892059.0

(51) Int. Cl. A61K 31/519; A61K 9/16; A61K 9/20; A61K 9/28

(52) U.S. Cl. CPC A61K 31/519; A61K 9/1694; A61K 9/2009; A61K 9/2013; A61K 9/2018; A61K 9/2027; A61K 9/288

(57) ABSTRACT

A ticagrelor solid dispersion, a method for preparing same, and use thereof. The ticagrelor solid dispersion includes the following components: (a) an active ingredient, comprising ticagrelor and/or a pharmaceutically acceptable salt thereof; (b) a carrier, including a pharmaceutically acceptable high-molecular-weight polymer; and (c) a surfactant, including a polyoxyethylene non-ionic surfactant. The ticagrelor solid dispersion has significantly improved dissolution and improved bioavailability.

### CROSS-REFERENCE TO RELATED APPLICATIONS

The present application is a national stage entry from International Application No. PCT/CN2023/095585, filed on May 22, 2023, published as International Publication No. WO 2024/021802 A1 on Feb. 1, 2024, and claims the priority benefit of the following Chinese patent application: Chinese patent application with application number: 202210892059.0, titled “ticagrelor solid dispersion, method for preparing same, and use thereof” filed on Jul. 27, 2022, of which the whole contents are incorporated by reference herein in their entireties.

### FIELD OF THE INVENTION

The present invention belongs to the field of pharmaceuticals, specifically relates to ticagrelor, or a solid dispersion of ticagrelor in form of pharmaceutically acceptable salt thereof, and a method for preparing the same and use thereof.

### BACKGROUND OF THE INVENTION

Ticagrelor, a platelet aggregation inhibitor, is a novel cyclopentyl triazolopyrimidine (CPTP) oral antiplatelet drug. Its chemical name is: (1S,2S,3R,5S)-3-[7-{[(1R,2S)-2-(3,4-difluorophenyl)cyclopropyl]amino}-5-(propylthio)-3H-[1,2,3]-triazolo[4,5-d]pyrimidin-3-yl]-5-(2-hydroxyethoxy)cyclopentane-1,2-diol, with the molecular formula of C₂₃H₂₈F₂N₆O₄S. The structural formula is as follows:

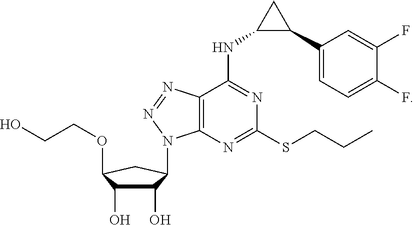

Ticagrelor is a member of the chemical classified cyclopentyl triazolopyrimidine (CPTP). CPTP is a selective adenosine diphosphate (ADP) receptor antagonist that acts at the P2Y12 ADP receptor to inhibit ADP-mediated platelet activation and aggregation by a mechanism of action similar to that of thienopyridines (e.g., clopidogrel). Unlike, however, the interaction between ticagrelor and the platelet P2Y12 ADP receptor is reversible, without conformational changes or signaling, and is followed by a rapid recovery of platelet function in the blood after discontinuation of the drug.

Ticagrelor tablets have been reported to have a bioavailability of approximately 36% and are marketed in 60 mg/90 mg specification, which are high in specification and low in bioavailability. According to the biopharmaceutical classification, ticagrelor belongs to BCS class 4, having low solubility and low permeability, which may be the primary reason for the low bioavailability of ticagrelor tablets.

Patent CN110876750A discloses a sustained-release composition of ticagrelor or a pharmaceutically acceptable salt thereof, in which the core granules of the sustained-release are solid dispersions of ticagrelor. As reproduced experimentally, the solid dispersions of ticagrelor involved in the patent had limited levels of dissolution improvement of ticagrelor and were unable to significantly increase its bioavailability.

Therefore, there is a need in the art for a solid dispersion of ticagrelor with significantly improved bioavailability.

### SUMMARY OF THE INVENTION

It is an object of the present application to provide a solid dispersion of ticagrelor, which is capable of significantly increasing the dissolution of ticagrelor and thus significantly increasing its bioavailability. In addition, the solid dispersion of the ticagrelor in the present application has good stability.

In a first aspect, the present application provides a solid dispersion of ticagrelor comprising the following components: (a) an active ingredient comprising ticagrelor and/or a pharmaceutically acceptable salt thereof; (b) a carrier comprising a pharmaceutically acceptable high-molecular-weight polymer; and (c) a surfactant comprising a polyoxyethylene-based non-ionic surfactant.

According to some embodiments of the present application, said surfactant comprises polyoxyethylene oleate glyceride and/or polyoxyethylene hydrogenated castor oil. In some embodiments, said surfactant comprises polyoxyethylene hydrogenated castor oil. Preferably, said surfactant is selected from at least one of polyoxyethylene hydrogenated castor oil 40 and polyoxyethylene hydrogenated castor oil 60. In some examples, said surfactant comprises or is polyoxyethylene hydrogenated castor oil 40. In some examples, said surfactant comprises or is polyoxyethylene hydrogenated castor oil 60.

According to some embodiments of the present application, the mass ratio of said active ingredient to said surfactant is 6:(2-10), for example, 6:2, 6:3, 6:4, 6:5, 6:6, 6:7, 6:8, 6:9, or 6:10. Preferably, the mass ratio of said active ingredient to said surfactant is 6:(2.5-8). Further preferably, the mass ratio of said active ingredient to said surfactant is 6:(3-7.2).

According to some embodiments of the present application, said high-molecular-weight polymer is selected from one or more of povidone, copovidone, hydroxypropyl methylcellulose, hydroxypropyl cellulose, hydroxyethyl cellulose, ethyl cellulose, polyvinyl caprolactam-polyvinyl acetate-polyglycol graft copolymer, polyoxyethylene, acrylic resins, polyvinyl acetate, and polyethylene glycol.

In the present application, the examples of povidone that may be used as a carrier include, but are not limited to, povidone K12, povidone K15, povidone K17, povidone K25, povidone K30, povidone K29/32, povidone K60, povidone K120, and the like, preferably povidone K29/32 and/or povidone K12.

In the present application, the examples of copovidone that may be used as a carrier include, but are not limited to, copovidone VA64, and copovidone S-630.

In the present application, hydroxypropyl methylcellulose that can be used as a carrier is classified in terms of viscosity as E3, E5, E6, E15, E50LV, and so on. In one example, hydroxypropyl methylcellulose is E5.

In the present application, the examples of polyethylene glycol that may be used as a carrier include, but are not limited to, PEG4000, PEG6000, and PEG8000. In some examples, the polyethylene glycol used as a carrier is polyethylene glycol 4000 or 6000.

According to some embodiments of the present application, the mass ratio of said active ingredient to said carrier is 1:(0.25-13), such as, 1:0.25, 1:0.50, 1:0.75, 1:1, 1:2, 1:2.5, 1:3, 1:4, 1:5, 1:6, 1:7, 1:8, 1:9, 1:10, 1:13, etc. preferably 1:(0.5-10), and most preferably 1:(1-8).

According to some embodiments of the present application, said solid dispersion further comprises an antioxidant, preferably said antioxidant is selected from one or more of butyl hydroxyanisole, dibutyl hydroxytoluene and vitamin E polyethylene glycol succinate. In one preferred embodiment, the antioxidant is butyl hydroxyanisole.

According to some embodiments of the present application, the mass ratio of said active ingredient to said antioxidant is 15:(0.02-0.4), such as 15:0.02, 15:0.04, 15:0.06, 15:0.08, 15:0.10, 15:0.12, or 15:0.15, more preferably 15:(0.02-0.17), and most preferably 15:(0.02-0.1).

According to some embodiments of the present application, said solid dispersion further comprises one or more of a filler, a disintegrant and a lubricant.

According to some embodiments of the present application, said filler is selected from one or more of microcrystalline cellulose, lactose, calcium hydrogen phosphate, and silicon dioxide.

According to some embodiments of the present application, said disintegrant is selected from one or more of cross-linked povidone, cross-linked sodium carboxymethyl cellulose, sodium carboxymethyl starch, and low-substituted hydroxypropyl cellulose.

According to some embodiments of the present application, said lubricant is selected from one or more of magnesium stearate, sodium stearate fumarate, stearic acid, polyethylene glycol (preferably having an average molecular weight of 4000 to 6000), talc, hydrogenated vegetable oil, and colloidal silicon dioxide.

According to some embodiments of the present application, the mass ratio of said active ingredient to said filler is 1:(0.5-6). preferably 1:(0.7-5), more preferably 1:(1.0-5). In some embodiments, the mass ratio of said active ingredient to said filler is 1:(0.79-4.84). In some embodiments, the mass ratio of said active ingredient to said filler is 1:0.7, 1:0.8, 1:1, 1:2, 1:3, 1:4 or 1:5. In some embodiments, the mass ratio of said active ingredient to said filler is 1:(1.24-4.84).

According to some embodiments of the present application, the mass ratio of said active ingredient to said disintegrant is 1:(0.20-1.5), preferably 1:(0.30-1.18). In some examples, the mass ratio of said active ingredient to said disintegrant is 1:0.30, 1:0.40, 1:0.50, 1:0.60, 1:0.70, 1:0.80, 1:0.90, 1:1.0, 1:1.1, or 1:1.15. According to some embodiments of the present application, the mass ratio of ticagrelor or a pharmaceutically acceptable salt thereof to said lubricant is 1:(0.02-0.04).

According to some embodiments of the present application, said filler comprises silicon dioxide and/or microcrystalline cellulose, preferably, said filler is silicon dioxide and microcrystalline cellulose, more preferably, the mass ratio of microcrystalline cellulose to silicon dioxide is (0.17-6.0): 1, such as, 0.2:1, 0.3:1, 0.5:1, 0.6:1, 0.64:10.8:1, 1:1, 1.2:1, 1.5:1, 1.8:1, 2:1, 2.5:1, 3.0:1, 3.5:1, 4.0:1, 4.5:1, 5:1, or 5.6:1. Further preferably, the mass ratio of microcrystalline cellulose to silicon dioxide is (0.64-5.6): 1.

According to some embodiments of the present application, said ticagrelor or pharmaceutically acceptable salt thereof is present in molecular, colloidal, microcrystalline, or amorphous form.

According to some embodiments of the present application, said solid dispersion is prepared by a method selected from the group consisting of vacuum drying, spray drying, hot-melt extrusion, wet granulation, and fluidized bed granulation, preferably, fluidized bed granulation. The one-step granulation method through fluidized bed produces granules with a narrow distribution range, uniformity, non-stickiness between granules and good compressibility, and has high industrial feasibility.

In a second aspect, the present application further provides a method for preparing the solid dispersion of ticagrelor as described above, comprising the following steps:

(1) dissolving a pharmaceutically acceptable high-molecular-weight polymer and a surfactant, and optionally an antioxidant, in a solvent, to provide a first mixture; (2) dissolving ticagrelor and/or a pharmaceutically acceptable salt thereof in said first mixture to provide a second mixture; and (3) spraying said second mixture onto a surface of a fluidized bed granulation substrate through a fluidized bed granulation process.

According to some embodiments of the present application, said fluidized bed granulation substrate is selected from one or more of microcrystalline cellulose, lactose, calcium hydrogen phosphate, and silicon dioxide.

According to some embodiments of the present application, the mass ratio of ticagrelor or a pharmaceutically acceptable salt thereof to said fluidized bed granulation substrate is 1:(0.6-3.6), such as 1:0.6, 1:0.7, 1:0.8, 1:1, 1:1.5, 1:2.0, 1:2.5, 1:3.0, or 1:3.5, more preferably 1:(0.7-3.0), and most preferably 1:(0.7-2.8). In some examples, the mass ratio of said ticagrelor or pharmaceutically acceptable salt thereof to said fluidized bed granulation substrate is 1:(0.75-2.5).

According to some embodiments of the present application, said fluidized bed granulation substrate comprises silicon dioxide and microcrystalline cellulose. Preferably, the mass ratio of silicon dioxide to microcrystalline cellulose of (13.0-17.5): (8.0-23.5) is suitable for one-step granulation, more preferably, the mass ratio of silicon dioxide to microcrystalline cellulose is (13.0-15.0): (8.0-10.0), further preferably (13.0-14.0): (8.0-9.0), such as 13.02:8.88 or 13: (8.0-9.0).

According to some embodiments of the present application, said solvent is an alcoholic solvent or an aqueous solution thereof, preferably a C1-C3 alcohol or an aqueous solution thereof, more preferably an aqueous solution of ethanol.

The granulation method of the present application produces granules with a narrow particle size distribution range, uniformity, non-stickiness between granules and good compressibility, and has high industrial feasibility.

In a third aspect, the present application provides a pharmaceutical formulation comprising a solid dispersion as described in the present application and a pharmaceutically acceptable excipient, wherein said pharmaceutical formulation is selected from a capsule, a dry suspension, a granule, a fine granule, or a tablet.

In some embodiments, said excipient in the pharmaceutical formulation may be one or more of a filler (e.g. silicon dioxide and/or microcrystalline cellulose), a disintegrant, and a lubricant. In some embodiments, the mass ratio of said active ingredient to said filler (e.g. silicon dioxide and/or microcrystalline cellulose) is 1:(2.47-4.84).

The present invention improves the dissolution and bioavailability of the drug by means of the solid dispersion prepared with a specific surfactant as well as a carrier.

### BRIEF DESCRIPTION OF THE DRAWINGS

FIG. 1 is an X-ray diffraction pattern of the solid dispersion granules of Prescription 34.

FIG. 2 shows the results of dissolution test for Prescription 34.

### DETAILED DESCRIPTION OF THE EMBODIMENTS

The invention is further described below in connection with the accompanying drawings and specific examples, the invention applies to the prior art where not already mentioned herein. Specific examples of the present invention are given below, but the examples are intended only to describe the present description in further detail and do not limit the invention.

Assay for release degree: This product was taken and tested according to the release degree assay (Chinese Pharmacopoeia 2020 Edition, Part IV, 0931, second method), the dissolution medium was a 0.1 M hydrochloric acid solution, and after sampling, the release degree assay was conducted using high-performance liquid chromatography. All the followings were tested and analyzed using this analytical method. Dissolution results were expressed as mean values (N=3).

X-ray pattern determination method: X-ray powder diffraction instrument: Shimadzu XRD-6100 X-ray powder diffractometer; Radiation source Cu (1.54060 A); Generator (Target) mA: 10 mA; Starting 2θ: 3°; Scanning range: 3° to 60°; Scanning steps: 0.02°; and Scanning speed: 2°/min.

The dissolution of ticagrelor tablets (90 mg; Lot: YADH; AstraZeneca) in 0.1 M hydrochloric acid is shown in Table 1 below:

TABLE 1
0.1M HCl, paddle process 75 rpm (cumulative release, %)

| Batch number | 5 min | 15 min | 30 min | 60 min |
|---|---|---|---|---|
| YADH | 5 | 6 | 7 | 8 |

Comparative example 1: Solid dispersion was prepared in reference to the patent CN110876750A.

Specific composition of the prescription is shown in Table 2:

TABLE 2

| | | 90 mg specification (%) |
|---|---|---|
| Functionality | Ingredient | Comparative example 1 |
| Active ingredient | Ticagrelor | 50.00 |
| Carrier | Copovidone | 50.00 |

Preparation process: Ticagrelor was dissolved with copovidone (the mass ratio of 1:1) in anhydrous ethanol, and prepared into a solid dispersion by spray drying.

The results of dissolution tests are shown in Table 3:

TABLE 3
0.1M HCl, paddle process 75 rpm (cumulative release, %)

| Batch | 5 min | 15 min | 30 min | 60 min |
|---|---|---|---|---|
| YADH | 5 | 6 | 7 | 8 |
| Comparative | 8 | 14 | 18 | 19 |
| example 1 | | | | |

The dissolution of the solid dispersion prepared in reference to patent CN110876750A in 0.1 M hydrochloric acid in 60 minutes is increased by more than one time compared to ticagrelor tablets.

### Example 1: Screening of Surfactants

Specific compositions of prescriptions 1 to 7 are shown in Table 4.

TABLE 4

| | | 90 mg specification (%) | | | | | | |
|---|---|---|---|---|---|---|---|---|
| | | Pre- | Pre- | Pre- | Pre- | Pre- | Pre- | Pre- |
| | | scription | scription | scription | scription | scription | scription | scription |
| Functionality | Ingredient | 1 | 2 | 3 | 4 | 5 | 6 | 7 |
| Active | Ticagrelor | 13.78 | 13.78 | 13.78 | 13.78 | 13.78 | 13.78 | 13.78 |
| ingredient | | | | | | | | |
| Carrier | Copovidone | 38.59 | 38.59 | 38.59 | 38.59 | 38.59 | 38.59 | 38.59 |
| | VA64 | | | | | | | |
| Surfactant | Caprylic/capric | 2.76 | — | — | — | — | — | — |
| | polyethylene | | | | | | | |
| | glycol | | | | | | | |
| | glyceride | | | | | | | |
| | Propylene | — | 2.76 | — | — | — | — | — |
| | glycol | | | | | | | |
| | monocaprylate | | | | | | | |
| | 90 | | | | | | | |
| | Polyglyceryl | — | — | 2.76 | — | — | — | — |
| | oleate | | | | | | | |
| | Diethylene | — | — | — | 2.76 | — | — | — |
| | glycol | | | | | | | |
| | monoethyl | | | | | | | |
| | ether HP | | | | | | | |
| | Medium chain | — | — | — | — | 2.76 | — | — |
| | triglyceride | | | | | | | |
| | Polyoxyethylene | — | — | — | — | — | 2.76 | — |
| | oleate | | | | | | | |
| | glycerides | | | | | | | |
| | Polyoxyethylene | — | — | — | — | — | — | 2.76 |
| | 40 | | | | | | | |
| | hydrogenated | | | | | | | |
| | castor oil | | | | | | | |
| Filler | Microcrystalline | 34.83 | 34.83 | 34.83 | 34.83 | 34.83 | 34.83 | 34.83 |
| | cellulose | | | | | | | |
| Disintegrant | Cross-linked | 9.65 | 9.65 | 9.65 | 9.65 | 9.65 | 9.65 | 9.65 |
| | povidone | | | | | | | |
| Lubricant | Magnesium | 0.39 | 0.39 | 0.39 | 0.39 | 0.39 | 0.39 | 0.39 |
| | stearate | | | | | | | |

### Preparation Process:

Prescription 1: Copovidone and caprylic/capric polyethylene glycol glyceride were taken and weighted according to the prescribed amounts, added sequentially to an aqueous solution of ethanol, and stirred until completely dissolved, followed by adding slowly the prescribed amount of ticagrelor and after the drug was dissolved until clear, drying in a vacuum drying oven in which the temperature was set as 40 to 60° C., and vacuum degree was −0.5 to −1.0 bar, to produce the solid dispersion granules. Then the solid dispersion granules were mixed with the prescribed amount of microcrystalline cellulose and cross-linked povidone to provide the mixed granules 1. The magnesium stearate was mixed well with about 3 times amount of the mixed granules 1, and the mixture was passed through a 40 mesh sieve and then mixed with the remaining mixed granules 1 well before filling into the capsule.

Prescriptions 2 to 7: The preparation process was the same as that of Prescription 1, the prescriptions differ only in the surfactant, wherein the surfactants in order of prescriptions 2 to 7 were: propylene glycol monocaprylate 90, polyglyceryl oleate, diethylene glycol monoethyl ether, medium chain triglycerides, polyoxyethylene oleate glycerides, and polyoxyethylene 40 hydrogenated castor oil, respectively.

The results of dissolution tests for Prescriptions 1 to 7 are shown in Table 5 below.

TABLE 5
0.1M HCl, paddle process 75 rpm (cumulative release, %)

| Batch | 5 min | 15 min | 30 min | 60 min |
|---|---|---|---|---|
| YADH | 5 | 6 | 7 | 8 |
| Comparative | 8 | 14 | 18 | 19 |
| example 1 | | | | |
| Prescription 1 | 20 | 22 | 22 | 23 |
| Prescription 2 | 15 | 16 | 17 | 17 |
| Prescription 3 | 18 | 20 | 21 | 21 |
| Prescription 4 | 18 | 20 | 22 | 22 |
| Prescription 5 | 19 | 19 | 19 | 20 |
| Prescription 6 | 35 | 35 | 38 | 40 |
| Prescription 7 | 40 | 41 | 45 | 50 |

### Result Analysis:

The dissolution of Comparative example 1 was increased approximately 1.4 times compared to ticagrelor tablets, and during the experiments of extensive screening of surfactants, it was unexpectedly discovered that by adding the surfactants, Polyoxyethylene oleate glycerides and polyoxyethylene 40 hydrogenated castor oil, the dissolution of ticagrelor in Prescriptions 6 and 7 can be significantly increased compared to Comparative example 1, wherein the dissolution of Prescription 6 was increased by more than one time after the addition of the surfactant Polyoxyethylene oleate glycerides; and the dissolution of Prescription 7 was increased by about 1.6 times after the addition of polyoxyethylene 40 hydrogenated castor oil. Ticagrelor, a BCS class 4 drug, has been shown to improve its bioavailability with increased dissolution. Therefore, the addition of polyoxyethylene oleate glycerides or polyoxyethylene 40 hydrogenated castor oil to the prescription is more promising to increase the bioavailability of formulation compared to Comparative example 1.

### Example 2: Screening of Carriers

Specific compositions of Prescriptions 8 to 13 are shown in Table 6.

TABLE 6

| | | 90 mg specification (%) | | | | | |
|---|---|---|---|---|---|---|---|
| | | Pre- | Pre- | Pre- | Pre- | Pre- | Pre- |
| | | scription | scription | scription | scription | scription | scription |
| Functionality | Ingredient | 8 | 9 | 10 | 11 | 12 | 13 |
| Active | Ticagrelor | 13.78 | 13.78 | 13.78 | 13.78 | 13.78 | 13.78 |
| ingredient | | | | | | | |
| Carrier | Polyvidone K29/32 | — | — | 38.59 | — | — | 38.59 |
| | Hydroxypropyl | — | — | — | 38.59 | — | — |
| | methylcellulose E5 | | | | | | |
| | Copovidone VA64 | — | — | — | — | 38.59 | — |
| | Povidone K12 | — | 38.59 | — | — | — | — |
| | PEG 4000 | — | — | — | — | — | 38.59 |
| | Hydroxypropyl | 38.59 | — | — | — | — | — |
| | methylcellulose | | | | | | |
| | acetate succinate | | | | | | |
| Surfactant | Polyoxyethylene 40 | 2.76 | 2.76 | 2.76 | 2.76 | 2.76 | 2.76 |
| | hydrogenated castor | | | | | | |
| | oil | | | | | | |
| Filler | Microcrystalline | 34.67 | 34.83 | 28.22 | 34.83 | 40.46 | 28.22 |
| | cellulose | | | | | | |
| Disintegrant | Cross-linked | 9.65 | 9.65 | 16.26 | 9.65 | 4.13 | 16.26 |
| | Povidone XL | | | | | | |
| Lubricant | Magnesium stearate | 0.55 | 0.39 | 0.39 | 0.39 | 0.28 | 0.39 |

### Preparation Process:

Prescription 8: Carrier, ticagrelor, and surfactant were taken and weighted according to the prescribed amounts, and mixed well sequentially. The solid dispersion was prepared through hot-melt extrusion preparation process (setting the feed rate at 20 to 150 rpm, screw speed at 50 to 300 rpm, and temperature in the temperature zone at 80 to 200° C.), and then pulverized by a needle pulverizer (setting the feed speed at 10 to 150 rpm, crushing speed at 15000 to 24000 rpm, and sieve mesh at 0.6 mm). The pulverized solid dispersion granules were mixed with the prescribed amounts of microcrystalline cellulose and cross-linked povidone to provide mixed granules 1. The magnesium stearate was mixed well with about 3 times amount of the mixed granules 1, and the mixture was passed through a 40 mesh sieve and then mixed with the remaining mixed granules 1 well before filling into the capsule.

Prescription 9: Carrier and surfactant were taken and weighted according to the prescribed amounts, added sequentially to an aqueous solution of ethanol, and stirred until completely dissolved, followed by adding slowly the prescribed amount of ticagrelor and after the drug was dissolved until clear, formulating into a binder. The solid dispersion granules were prepared by spraying the binder onto microcrystalline cellulose through a fluidized bed granulation process (setting the inlet air temperature at 40 to 80° C., inlet air volume at 20 to 100 m³/h, and spraying speed at 3 to 10 g/min). Then the solid dispersion granules were mixed with the prescribed amount of cross-linked povidone to obtain mixed granules 1. The magnesium stearate was mixed well with about 3 times amount of the mixed granules 1, and the mixture was passed through a 40 mesh sieve and then mixed with the remaining mixed granules 1 well before filling into the capsule.

Prescription 10/13: Carrier and surfactant were taken and weighted according to the prescribed amounts, added sequentially to an aqueous solution of ethanol, and stirred until completely dissolved, followed by adding slowly the prescribed amount of ticagrelor and after the drug was dissolved until clear, drying in a vacuum drying oven in which the temperature was set as 40 to 60° C., and vacuum degree was −0.5 to −1.0 bar, to produce the solid dispersion granules. The solid dispersion granules were mixed with the prescribed amounts of microcrystalline cellulose and cross-linked povidone to provide the mixed granules 1. The magnesium stearate was mixed well with about 3 times amount of the mixed granules 1, and the mixture was passed through a 40 mesh sieve and then mixed with the remaining mixed granules 1 well to before filling into capsule.

Prescription 11: Carrier and surfactant were weighted according to the prescribed amounts, added sequentially to an aqueous solution of ethanol, and stirred until completely dissolved, followed by adding slowly the prescribed amount of ticagrelor and after the drug was dissolved until clear, drying in a vacuum drying oven in which the temperature was set as 40 to 60° C., and vacuum degree was −0.5 to −1.0 bar, to produce the solid dispersion granules. Then the solid dispersion granules were mixed with the prescribed amount of microcrystalline cellulose well and granulated by using a dry granulator (setting feed speed at 20 to 100 rpm, press wheel speed at 5 to 20 rpm, press wheel pressure at 15 to 45 bar, and pulverizer speed at 1000 to 2000 rpm), and then the granules were mixed with the prescribed amount of cross-linked povidone to provide mixed granules 1. The magnesium stearate was mixed well with about 3 times amount of the mixed granules 1, and the mixture was passed through a 40 mesh sieve and then mixed with the remaining mixed granules 1 well, and the powders were pressed into tablets directly.

Prescription 12: Carrier and surfactant were taken and weighted according to the prescribed amounts, added sequentially to an aqueous solution of ethanol, and stirred until completely dissolved, followed by adding slowly the prescribed amount of ticagrelor and after the drug was dissolved until clear, formulating into a binder. The binder was sprayed onto microcrystalline cellulose through a wet granulation process (setting the paddle speed at 50 to 300 rpm, the cutter speed at 1000 to 2000 rpm, and spraying speed at 15 to 100 g/min). The resulted wet granules were placed in a fluidized bed (setting the inlet air temperature at 40 to 80° C. and the inlet air volume at 20 to 100 m³/h) for drying. And the dry granules were granulated with 0.5 mm mesh sieve. After granulation, the granules were then mixed with the prescribed amount of cross-linked povidone to obtain mixed granules 1. The magnesium stearate was mixed well with about 3 times amount of the mixed granules 1, and the mixture was passed through a 40 mesh sieve and then mixed with the remaining mixed granules 1 well to before filling into capsule.

The results of dissolution tests for Prescriptions 8 to 13 are shown in Table 7 below.

TABLE 7
0.1M HCl, paddle process 75 rpm (cumulative release, %)

| Batch | 5 min | 15 min | 30 min | 60 min |
|---|---|---|---|---|
| YADH | 5 | 6 | 7 | 8 |
| Comparative | 8 | 14 | 18 | 19 |
| example 1 | | | | |
| Prescription 8 | 7 | 7 | 8 | 8 |
| Prescription 9 | 31 | 34 | 35 | 35 |
| Prescription 10 | 32 | 33 | 33 | 33 |
| Prescription 11 | 31 | 33 | 34 | 36 |
| Prescription 12 | 34 | 34 | 35 | 37 |
| Prescription 13 | 32 | 33 | 34 | 34 |

### Result Analysis:

The dissolution of Comparative example 1 was increased approximately 1.4 times compared to ticagrelor tablets, and during the experiments of extensive screening of carriers, it was unexpectedly discovered that after adding hydroxypropyl methylcellulose acetate succinate, Prescription 8 did not improve the dissolution of ticagrelor compared to Comparative example 1, however, by adding povidone, hydroxypropyl methylcellulose, and polyethylene glycol, Prescriptions 9 to 13 significantly improved the dissolution of ticagrelor compared to Comparative example 1, with the dissolution of Prescriptions 9 to 13 being increased by more than 0.5 times. Ticagrelor, a BCS class 4 drug, has been shown to improve its bioavailability with increased dissolution. Therefore, the addition of hydrophilic carriers to the prescription is more promising to increase the bioavailability of formulation compared to Comparative example 1.

### Example 3: Screening of the Ratio of Ticagrelor to Carrier

Specific ingredients of Prescriptions 14 to 21 are shown in Table 8 below.

TABLE 8

| | | 90 mg specification (%) | | | | | | | |
|---|---|---|---|---|---|---|---|---|---|
| | | Pre- | Pre- | Pre- | Pre- | Pre- | Pre- | Pre- | Pre- |
| | | scription | scription | scription | scription | scription | scription | scription | scription |
| Functionality | Ingredient | 14 | 15 | 16 | 17 | 18 | 19 | 20 | 21 |
| Active | Ticagrelor | 24.00 | 21.43 | 17.65 | 14.99 | 10.34 | 12.50 | 7.82 | 5.62 |
| ingredient | | | | | | | | | |
| Carrier | Copovidone | 3.60 | 6.43 | 10.59 | 13.51 | 18.62 | 27.08 | 37.55 | 43.82 |
| | VA64 | | | | | | | | |
| | Hydroxypropyl | 2.40 | 4.29 | 7.06 | 9.00 | 12.42 | — | 25.03 | 29.21 |
| | methylcellulose | | | | | | | | |
| | E5 | | | | | | | | |
| Surfactant | Polyoxyethylene | 20.00 | 17.86 | 14.71 | 12.50 | 8.62 | 10.42 | 6.52 | 4.68 |
| | 40 hydrogenated | | | | | | | | |
| | castor oil | | | | | | | | |
| Filler | Microcrystalline | 50.00 | 50.00 | 50.00 | 50.00 | 50.00 | 50.00 | 23.08 | 16.67 |
| | cellulose | | | | | | | | |

### Preparation Process:

Prescriptions 14 to 18 and 20 to 21: Copovidone, hydroxypropyl methylcellulose, and polyoxyethylene 40 hydrogenated castor oil were taken and weighted according to the prescribed amounts, added sequentially to an aqueous solution of ethanol, and stirred until completely dissolved, followed by adding slowly the prescribed amount of ticagrelor and after the drug was dissolved until clear, preparing the solid dispersion granules through a fluidized bed granulation process and filling into capsules.

Prescription 19: Copovidone and polyoxyethylene 40 hydrogenated castor oil were taken and weighted according to the prescribed amounts, added sequentially to an aqueous solution of ethanol, and stirred until completely dissolved, followed by adding slowly the prescribed amount of ticagrelor and after the drug was dissolved until clear, preparing the solid dispersion granules through a fluidized bed granulation process and directly pressing into tablets.

The results of dissolution tests for Prescriptions 14 to 21 are shown in Table 9 below.

TABLE 9
0.1M HCl, paddle process 75 rpm (cumulative release, %)

| Batch | 5 min | 15 min | 30 min | 60 min |
|---|---|---|---|---|
| YADH | 5 | 6 | 7 | 8 |
| Comparative | 8 | 14 | 18 | 19 |
| example 1 | | | | |
| 14 | 31 | 31 | 32 | 33 |
| 15 | 36 | 37 | 38 | 40 |
| 16 | 70 | 78 | 78 | 78 |
| 17 | 40 | 67 | 77 | 82 |
| 18 | 74 | 80 | 84 | 86 |
| 19 | 34 | 34 | 35 | 37 |
| 20 | 87 | 87 | 87 | 90 |
| 21 | 92 | 95 | 97 | 99 |

### Result Analysis:

The dissolution of Comparative example 1 was increased approximately 1.4 times compared to ticagrelor tablets, and by controlling the ratio of drug to carrier in the range of 1:0.25-13, Prescriptions 14 to 21 were able to significantly increase the dissolution of ticagrelor compared to Comparative example 1, their dissolutions were increased by more than one time compared to Comparative example 1. As the ratio of drug to carrier was increased, the dissolution also increased, wherein the dissolution was increased by more than approximately one time (Prescriptions 14 to 21), the dissolution was increased by more than three times (Prescription 17/18/20/21), and the dissolution was increased by even more than four times (Prescription 21). Combining the feasibility of formulation molding and industrialization considerations, the ratio of drug to carrier was preferably 1:0.25-13, more preferably 1:0.5-10, most preferably 1:1-8.

### Example 4: Screening of the Ratio of Surfactant

Specific ingredients of Prescriptions 22 to 28 are shown in Table 10 below.

TABLE 10

| | | 90 mg specification (%) | | | | | | |
|---|---|---|---|---|---|---|---|---|
| | | Pre- | Pre- | Pre- | Pre- | Pre- | Pre- | Pre- |
| | | scription | scription | scription | scription | scription | scription | scription |
| Functionality | Ingredient | 22 | 23 | 24 | 25 | 26 | 27 | 28 |
| Active | Ticagrelor | 24.00 | 23.08 | 21.43 | 20.00 | 17.65 | 16.67 | 15.79 |
| ingredient | | | | | | | | |
| Carrier | Copovidone | 14.40 | 13.85 | 12.86 | 12.00 | 10.59 | 10.00 | 9.47 |
| | VA64 | | | | | | | |
| | Hydroxypropyl | 9.60 | 9.23 | 8.57 | 8.00 | 7.06 | 6.67 | 6.32 |
| | methylcellulose | | | | | | | |
| | E5 | | | | | | | |
| Surfactant | Polyoxyethylene | 2.00 | 3.85 | 7.14 | 10.00 | 14.71 | 16.67 | 18.42 |
| | 40 hydrogenated | | | | | | | |
| | castor oil | | | | | | | |
| Filler | Microcrystalline | 50.00 | 50.00 | 50.00 | 50.00 | 50.00 | 50.00 | 50.00 |
| | cellulose | | | | | | | |

Preparation process: Copovidone, hydroxypropyl methyl cellulose, and polyoxyethylene 40 hydrogenated castor oil were taken and weighted according to the prescribed amounts, added sequentially to an aqueous solution of ethanol, and stirred until completely dissolved. The prescribed amount of ticagrelor was slowly added, and after the drug was dissolved until clear, the solid dispersion granules were prepared through a fluidized bed granulation process, and filled into capsules.

The results of dissolution tests are shown in Table 11 below.

TABLE 11
0.1M HCl, paddle process 75 rpm (cumulative release, %)

| Batch | 5 min | 15 min | 30 min | 60 min |
|---|---|---|---|---|
| YADH | 5 | 6 | 7 | 8 |
| Comparative | 8 | 14 | 18 | 19 |
| example 1 | | | | |
| 22 | 7 | 9 | 10 | 12 |
| 23 | 6 | 12 | 15 | 17 |
| 24 | 9 | 23 | 28 | 30 |
| 25 | 22 | 43 | 50 | 51 |
| 26 | 46 | 77 | 88 | 91 |
| 27 | 58 | 88 | 93 | 95 |
| 28 | 69 | 90 | 97 | 98 |

### Result Analysis:

The dissolution of Comparative example 1 was increased approximately 1.4 times compared to ticagrelor tablets, and during the experiments of extensive screening, it was discovered that, when the ratio of drug to surfactant was in the range of 6:0.5-1, Prescription 22/23 did not significantly increase the dissolution of ticagrelor compared to Comparative example 1, while the ratio of drug to surfactant was in the range of 6:2-7, Prescriptions 24 to 28 were able to significantly increase the dissolution of ticagrelor compared to Comparative example 1. As the ratio of drug to surfactant was increased, the dissolution also increased, wherein the dissolution was increased by more than 0.5 times (Prescriptions 24 to 28), the dissolution was increased by more than approximately 2 times (Prescriptions 25 to 28), and the dissolution was increased by even more than four times (Prescription 28). Combining with the feasibility of industrialization, the ratio of drug to surfactant was preferably 6:2-10, more preferably 6:2.5-8, most preferably 6:3-7.2.

### Example 5: Investigation of Fluidized Bed Substrate

Prescriptions 29 to 33 are shown in Table 12 below.

TABLE 12

| | | | 90 mg specification (%) | | | | |
|---|---|---|---|---|---|---|---|
| | | | Pre- | Pre- | Pre- | Pre- | Pre- |
| | | | scription | scription | scription | scription | scription |
| Functionality | | Ingredient | 29 | 30 | 31 | 32 | 33 |
| Active ingredient | | Ticagrelor | 18.85 | 18.85 | 24.57 | 21.69 | 27.57 |
| Carrier | Binder | Copovidone VA64 | 11.31 | 11.31 | 14.74 | 13.01 | 16.54 |
| | | Hydroxypropyl methylcellulose E5 | 7.54 | 7.54 | 9.83 | 8.67 | 11.03 |
| | | Polyoxyethylene 40 hydrogenated | 15.71 | 15.71 | 20.48 | 18.07 | 22.97 |
| | | castor oil | | | | | |
| | Substrate | silicon dioxide | 26.18 | — | 17.06 | 15.06 | 13.02 |
| | | Lactose monohydrate | — | 26.18 | — | — | — |
| | | Microcrystalline cellulose | 20.42 | 20.42 | 13.31 | 23.49 | 8.88 |

Preparation process: Hydroxypropyl methylcellulose, copovidone, and polyoxyethylene 40 hydrogenated castor oil were taken and weighted according to the prescribed amounts, added sequentially to an aqueous solution of ethanol, and stirred until completely dissolved, followed by adding slowly the prescribed amount of ticagrelor and after the drug was dissolved until clear, formulating into a binder. The solid dispersion granules were prepared by spraying the binder onto a mixture of silicon dioxide and/or lactose monohydrate and/or microcrystalline cellulose (setting the inlet air temperature at 40 to 80° C., inlet air volume at 20 to 100 m³/h, and spraying speed at 3 to 10 g/min) through a fluidized bed granulation process.

The results of the granulation showed that the oil wetness of Prescription 30 (without silicon dioxide) is higher than that of Prescription 29, the granules were easily bonded, seriously affecting the preparation of downstream processes (e.g., substandard mixing uniformity of granules, sticky flushing of pressed tablets); the excessive amount of substrate used in prescription 29 led to an over-sized formulation, which in turn would result in over-sized tablets and capsules, which is not conducive to patient administration and increases production costs, and the homogeneity of the formulations obtained by the preparation of Prescriptions 31 to 33 was good, without sticky flushing of pressed tablets. Therefore, it is preferred that silicon dioxide and microcrystalline cellulose are used as the substrate and the ratio is screened with silicon dioxide:microcrystalline cellulose of (13.00˜17.5): (8.0˜23.5), for example, (13.02-17.06): (8.88-13.31) is suitable for one-step granulation, and the preferable ratio of silicon dioxide to microcrystalline cellulose is (13.0-15.0): (8.0-10.0), or (13.0-14.0): (8.0-9.0), such as 13.02:8.88.

Particle size distribution of granules of Prescription 33 by the fluidized bed granulation is shown in Table 13.

TABLE 13

| Particle size (in microns) | Interval distribution % |
|---|---|
| 0-75 | 3.6 |
| 75-125 | 8.4 |
| 125-150 | 4.9 |
| 150-180 | 6.6 |
| 180-250 | 19.2 |
| 250-355 | 48.5 |
| 355-850 | 8.8 |
| >850 | 0 |

The one-step granulation process through fluidized bed produces granules with a narrow distribution range, uniformity, non-stickiness between granules and good compressibility, and has high industrial feasibility.

### Example 6

Formulation of Prescription 34 is shown in Table 14.

TABLE 14

| | | 90 mg |
|---|---|---|
| | | specification (%) |
| Functionality | Ingredient | Prescription 34 |
| Solid | Ticagrelor | 15.00 |
| dispersion of | Copovidone VA64 | 9.00 |
| Ticagrelor | Hydroxypropyl | 6.00 |
| | methylcellulose E5 | |
| | Polyoxyethylene 40 | 12.50 |
| | hydrogenated castor oil | |
| | Butyl hydroxyanisole | 0.02 |
| | silicon dioxide | 7.08 |
| | Microcrystalline cellulose | 4.83 |
| Filler | Microcrystalline cellulose | 34.83 |
| Disintegrant | Cross-linked povidone | 10.32 |
| Lubricant | Magnesium stearate | 0.42 |
| Coating | Opadry | Naked tablets |
| powder | | with 3% |
| | | weight gain |

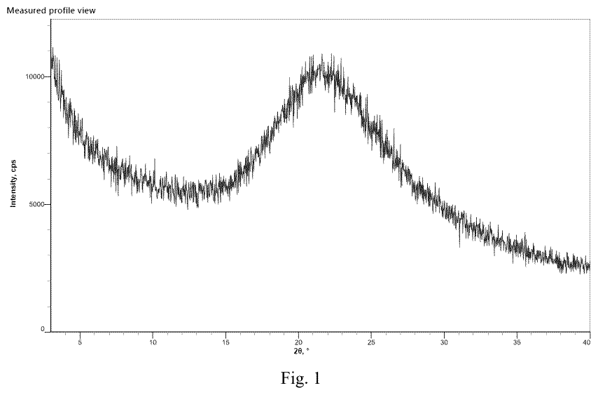

Preparation process: Copovidone, hydroxypropyl methyl cellulose, polyoxyethylene 40 hydrogenated castor oil, and butyl hydroxyanisole were taken and weighted according to the prescribed amounts, added sequentially to an aqueous solution of ethanol, and stirred until completely dissolved, followed by adding slowly the prescribed amount of ticagrelor and after the drug was dissolved until clear, formulating into a binder. The solid dispersion granules were prepared by spraying the binder onto a mixture of silicon dioxide and microcrystalline cellulose (setting the inlet air temperature at 40 to 80° C., inlet air volume at 20 to 100 m³/h, and spraying speed at 3 to 10 g/min) through a fluidized bed granulation process. Its X-ray diffraction pattern was shown in FIG. 1, showing that ticagrelor in solid dispersion was amorphous.

The prepared solid dispersion granules were mixed with the prescribed amounts of microcrystalline cellulose and cross-linked povidone well to obtain mixed granules 1. The magnesium stearate was mixed well with about 3 times amount of the mixed granules 1, the mixture was passed through a 40 mesh sieve and then mixed with the remaining mixed granules 1 well, and the powders were pressed into tablets directly and coated with Opadry coating powder.

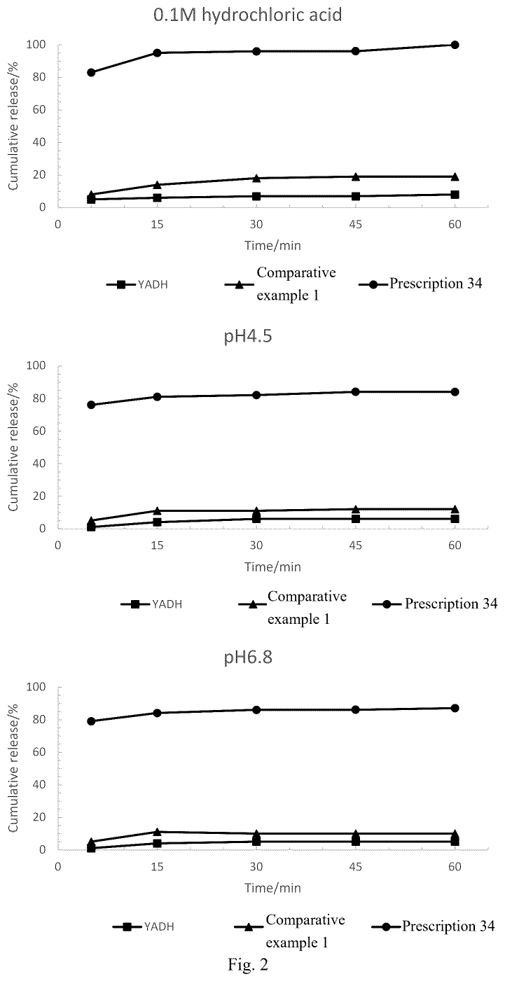

The results of the dissolution tests are shown in FIG. 2 and Tables 15 to 17.

TABLE 15
0.1M HCl, paddle process 75 rpm (cumulative release, %)

| Batch | 5 min | 15 min | 30 min | 45 min | 60 min |
|---|---|---|---|---|---|
| YADH | 5 | 6 | 7 | 7 | 8 |
| Comparative | 8 | 14 | 18 | 19 | 19 |
| example 1 | | | | | |
| Prescription | 83 | 95 | 96 | 96 | 100 |
| 34 | | | | | |

TABLE 16
pH 4.5, paddle process 75 rpm (cumulative release, %)

| Batch | 5 min | 15 min | 30 min | 45 min | 60 min |
|---|---|---|---|---|---|
| YADH | 1 | 4 | 6 | 6 | 6 |
| Comparative | 5 | 11 | 11 | 12 | 12 |
| example 1 | | | | | |
| Prescription | 76 | 81 | 82 | 84 | 84 |
| 34 | | | | | |

TABLE 17
pH 6.8, paddle process 75 rpm (cumulative release, %)

| Batch | 5 min | 15 min | 30 min | 45 min | 60 min |
|---|---|---|---|---|---|
| YADH | 1 | 4 | 5 | 5 | 5 |
| Comparative | 5 | 11 | 10 | 10 | 10 |
| example 1 | | | | | |
| Prescription | 79 | 84 | 86 | 86 | 87 |
| 34 | | | | | |

### Result Analysis:

In medium of 0.1 M hydrochloric acid, pH 4.5, or pH 6.8, the dissolution of Comparative example 1 was increased by about 1 time compared to ticagrelor tablets, whereas the dissolution of the formulation of Prescription 34 was significantly higher than that of Comparative example 1, the dissolution was significantly increased by more than about 4 times, wherein the dissolution in 0.1 M hydrochloric acid was increased by more than 4 times compared to Comparative example 1, the dissolution at pH 4.5 was increased by more than 6 times compared to Comparative example 1, and the dissolution at pH 6.8 was increased by more than about 8 times compared to Comparative example 1. Ticagrelor, a BCS class 4 drug, has been shown to improve its bioavailability with increased dissolution. Therefore, Prescription 34 is more promising to improve in vivo bioavailability than that of Comparative example 1 over ticagrelor tables.

### Example 7: Investigation of Stability of Prescription 34

The results of accelerated dissolution stability tests of Prescription 34 are shown in Table 18.

TABLE 18
0.1M HCl, paddle process 75 rpm 900 mL (cumulative release, %)

| Time | 5 min | 15 min | 30 min | 60 min |
|---|---|---|---|---|
| Day 0 | 83 | 95 | 96 | 100 |
| Acceleration | 84 | 95 | 98 | 99 |
| for 1 month | | | | |
| Acceleration | 85 | 96 | 97 | 98 |
| for 3 months | | | | |
| Acceleration | 86 | 94 | 93 | 100 |
| for 6 months | | | | |

Results analysis: there was no significant change in dissolution of Prescription 34 in hydrochloric acid after accelerating for 6 months compared to day 0.

The results of the accelerated test for the content of the substance of interest in Prescription 34 are shown in Table 19.

TABLE 19

| | | | | Acceleration | Acceleration | Acceleration |
|---|---|---|---|---|---|---|
| Time | | Day 0 | | for 1 month | for 3 months | for 6 months |
| Substance of interest | Acceptability criteria | / | | / | / | / |
| | Impurities A % | ≤0.2% | 0.04 | 0.05 | 0.04 | 0.04 |
| | Oxidized impurities 1% | ≤0.2% | 0.04 | 0.04 | 0.04 | 0.05 |
| | Oxidized impurities 2% | ≤0.2% | 0.04 | 0.05 | 0.05 | 0.04 |
| | Unknown impurities % | ≤0.2% | 0.03 | 0.03 | 0.03 | 0.03 |
| | (maximum) | | | | | |
| | Total impurities % | ≤0.5% | 0.15 | 0.17 | 0.16 | 0.16 |

Results analysis: There was no significant change in the substance of interest of Prescription 34 after accelerating for 6 months compared to day 0.

Experiments evidenced that the solid dispersions of ticagrelor of the present application, as well as the formulations prepared therefrom, have a high dissolution, and thus have good bioavailability.

1. A solid dispersion of ticagrelor, comprising the following components:
(a) an active ingredient comprising ticagrelor and/or a pharmaceutically acceptable salt thereof;
(b) a carrier comprising a pharmaceutically acceptable high-molecular-weight polymer; and
(c) a surfactant comprising a polyoxyethylene-based non-ionic surfactant.

2. The solid dispersion according to claim 1, characterized in that, the surfactant is selected from one or more of polyoxyethylene oleate glyceride and polyoxyethylene hydrogenated castor oil, preferably the surfactant comprises polyoxyethylene hydrogenated castor oil 40 and/or polyoxyethylene hydrogenated castor oil 60.

3. The solid dispersion according to claim 1, characterized in that, the mass ratio of the active ingredient to the surfactant is 6:(2-10), more preferably 6:(2.5-8), and most preferably 6:(3-7.2).

4. The solid dispersion according to claim 1, characterized in that, the high-molecular-weight polymer is selected from one or more of povidone, copovidone, hydroxypropyl methylcellulose, hydroxypropyl cellulose, hydroxyethyl cellulose, ethyl cellulose, polyvinyl caprolactam-polyvinyl acetate-polyglycol graft copolymer, polyoxyethylene, acrylic resins, polyvinyl acetate, and polyethylene glycol.

5. The solid dispersion according to claim 1, characterized in that, the mass ratio of the active ingredient to the carrier is 1:(0.25-13), more preferably 1:(0.5-10), and most preferably 1:(1-8).

6. The solid dispersion according to claim 1, characterized in that, further comprising an antioxidant,
preferably, the antioxidant is selected from one or more of butyl hydroxyanisole, dibutyl hydroxy toluene and vitamin E polyethylene glycol succinate, and
preferably, the mass ratio of ticagrelor or a pharmaceutically acceptable salt thereof to the antioxidant is 15:(0.02-0.4), more preferably 15:(0.02-0.17), and most preferably 15:(0.02-0.1).

7. The solid dispersion according to claim 1, further comprising one or more of a filler, a disintegrant, and a lubricant;
preferably, the filler is selected from one or more of microcrystalline cellulose, lactose, calcium hydrogen phosphate, and silicon dioxide; and/or
preferably, the disintegrant is selected from one or more of cross-linked povidone, cross-linked sodium carboxymethyl cellulose, sodium carboxymethyl starch, and low-substituted hydroxypropyl cellulose; and/or
preferably, the lubricant is selected from one or more of magnesium stearate, sodium stearate fumarate, stearic acid, polyethylene glycol, talc, hydrogenated vegetable oil, and colloidal silicon dioxide.

8. The solid dispersion according to claim 6, characterized in that,
the mass ratio of the active ingredient to the filler is 1:(0.5-6), preferably 1:(0.7-5), and more preferably 1:(1.0-5);
the mass ratio of the active ingredient to the disintegrant is 1:(0.30-1.18); and/or
the mass ratio of the active ingredient to the lubricant is 1:(0.02-0.04).

9. The solid dispersion according to claim 8, characterized in that, the filler comprises silicon dioxide and/or microcrystalline cellulose, preferably the filler is silicon dioxide and microcrystalline cellulose, more preferably the mass ratio of microcrystalline cellulose to silicon dioxide is (0.17-6.0): 1, and further preferably the mass ratio of microcrystalline cellulose to silicon dioxide is (0.64-5.6): 1.

10. The solid dispersion according to claim 1, characterized in that, the active ingredient is present in molecular, colloidal, microcrystalline, or amorphous form.

11. The solid dispersion according to claim 1, characterized in that, the solid dispersion is prepared by a method selected from the group consisting of vacuum drying, spray drying, hot-melt extrusion, wet granulation, and fluidized bed granulation, preferably, fluidized bed granulation.

12. A method for preparing the solid dispersion of ticagrelor according to claim 1, comprising the following steps:
(1) dissolving a pharmaceutically acceptable high-molecular-weight polymer and a surfactant in a solvent to provide a first mixture;
(2) dissolving an active ingredient in the first mixture to provide a second mixture; and
(3) spraying the second mixture onto a surface of a fluidized bed granulation substrate through a fluidized bed granulation process,
preferably, the mass ratio of ticagrelor or a pharmaceutically acceptable salt thereof to the fluidized bed granulation substrate is 1:(0.6-3.6), more preferably 1:(0.7-3.0), and most preferably 1:(0.7-2.8); and
preferably, the fluidized bed granulation substrate comprises silicon dioxide and microcrystalline cellulose, more preferably in the fluidized bed granulation substrate, the mass ratio of silicon dioxide to microcrystalline cellulose is (13.0-17.5): (8.0-23.5), (13.0-15.0): (8.0-10.0), or (13.0-14.0): (8.0-9.0).

13. A pharmaceutical formulation, comprising the solid dispersion according to claim 1 and a pharmaceutically acceptable excipient, wherein the pharmaceutical formulation is a capsule, a dry suspension, a granule, a fine granule, or a tablet.
